(19) United States
(12) Patent Application Publication
(10) Pub. No.: US 2018/0025033 A1
(43) Pub. Date: Jan. 25, 2018

(54) COMPUTER ARCHITECTURE FOR CREATING MULTIVARIATE PREDICTIVE MODELS OF OYSTER POPULATIONS

(71) Applicant: United States of America as Represented by The Secretary of The Army, Alexandria, VA (US)

(72) Inventors: Michael Kjelland, Vicksburg, MS (US); Todd M. Swannack, Austin, TX (US); Candice D. Piercy, Vicksburg, MS (US)

(21) Appl. No.: 15/479,132

(22) Filed: Apr. 4, 2017

Related U.S. Application Data
(60) Provisional application No. 62/365,726, filed on Jul. 22, 2016.

(51) Int. Cl. G06F 17/30; G06N 7/04; G06F 17/28; G06F 17/50; G06F 17/40; G06F 17/18

(52) U.S. Cl. CPC G06F 17/30294; G06F 17/40; G06F 17/18; G06F 17/2818; G06F 17/5009; G06N 7/04; A61B 2503/40

(57) ABSTRACT

A Multivariate Predictive Modeling System that determines the impact of numerous environmental, life-cycle, and policy-based variables on oyster populations in real time by instantiating Oyster Group Demographic Objects and Reef Objects which function as virtual processing components. The Multivariate Predictive Modeling System creates novel interactive digital replicas of oyster population and reef entities which may be updated in real time to model environmental impacts on oyster population growth.

### CROSS-REFERENCE TO RELATED APPLICATIONS

This patent application claims the benefit of U.S. Provisional Application No. 62/365,726 filed Jul. 22, 2016.

### STATEMENT REGARDING FEDERALLY SPONSORED RESEARCH OR DEVELOPMENT

The invention described herein was made by an employee of the United States Government and may be manufactured and used by the Government of the United States of America for governmental purposes without the payment of any royalties thereon or therefore.

### FIELD OF INVENTION

This invention relates to the field of computer processing architecture, and specifically to a method for creating interactive digital replicas (model) of oyster and reef entities which may be altered and updated in real time to create novel Multivariate Predictive Models of environmental impacts on oyster population growth.

### BACKGROUND OF THE INVENTION

Over $10 billion of seafood products are processed in the U.S. each year; oysters have traditionally been a significant component. In the 1970's, one-third of all U.S. fisheries produced oyster-related products, employing residents in 48 states. Since the 1990's, oyster harvests have dropped more than 85%, displacing local employees and increasing the U.S. foreign trade deficit as imported food products have been substituted. The U.S. foreign trade deficit for seafood, which is second only to crude oil, has increased dramatically as a result of diminishing oyster harvests.

More than twenty federal agencies and nine state agencies are currently undertaking oyster restoration research projects expected to total more than $500 million dollars. Private sector companies are investing heavily in acquaculature.

Federal agencies involved in oyster restoration projects include the U.S. Army Corps of Engineers (USACE), National Oceanic and Atmospheric Administration (NOAA), Department of Defense (DOD), National Park Service (NPS), U.S. Department of Agriculture (USDA), U.S. Department of Defense (DOD), U.S. Department of Homeland Security (DHS), U.S. Department of the Interior, U.S. Federal Highway Administration (FHA), U.S. Fish and Wildlife Service (USFWS) and U.S. Forest Service (USFS).

The largest collaborative project is the Chesapeake Bay Project (CBP). The CBP is a comprehensive study of ten major oyster reefs located in Maryland, Virginia, Pennsylvania and Vermont. The CBP tracks hundreds of variables related to currents, temperature, salinity, and Total Suspended Solids (TSS), which impacted reefs and the timing of harvests relative to the survival of larvae and juveniles at various critical life stages.

Because the CBP project parameters and other ecosystems under study are too large and complex for direct monitoring, scientist and researchers rely on computer modeling and simulation tools. These systems statistically extrapolate and predict environmental conditions and predict impacts. Increasingly powerful models simulate current state data and are used to predict future impacts on future oyster populations under different scenarios.

Research is directed at creating globally relevant and statistically accurate predictive models under alternative scenarios. However, models under various studies may be produced using different protocols, and may measure different parameters. Researchers continually attempt to reduce the error associated with models, and to apply knowledge gained from previous studies.

There is an unmet need for computer modeling tools which allow researchers to access, adapt, combine and standardize statistical methodologies for future predictive oyster population models.

There is an unmet need for modeling tools which allow rapid comparison and extrapolation of data and identification of relationships.

There is a further unmet need for a specialized modeling tool that can produce multiple highly complex, Multivariate Predictive Models in real time.

### BRIEF SUMMARY OF THE INVENTION

The invention is a specialized computer architecture for creating Multivariate Predictive Models of oyster populations within reefs. In various embodiments, the computer architecture includes one or more virtual machines, each of which include a Project Class, Reef Class and an Oyster Group Demographic Classes. The Project Class receives geographical parameters, time parameters and reef association parameters to instantiate at least one Project which includes digital replicas of oyster demographic groups and reefs within an area under study.

Each Reef Class is a virtual machine configured with processing functions to instantiate Reef Objects, Reef Objects are virtual machines defined by reef attributes with corresponding values, and Reef Object functions which are invoked when attribute values are instantiated or updated.

Each Oyster Group Demographic Class is a virtual machine configured with processing functions to instantiate Oyster Group Demographic Objects. Oyster Group Demographic Objects are virtual machines which have attributes with corresponding values and functions to represent oyster demographic groups associated with Reef Objects. Processing functions are invoked when attribute values are instantiated or updated.

### BRIEF DESCRIPTION OF THE DRAWING(S)

FIG. 1a illustrates an exemplary Multivariate Predictive Modeling System architecture.

FIG. 1b illustrates geographically distributed embodiment of a Multivariate Predictive Modeling System which is remotely accessed by multiple users to create Multivariate Predictive Models.

FIG. 2 illustrates an exemplary method for creating Multivariate Oyster Population Model reflecting an initial baseline state.

FIG. 3 illustrates an exemplary method for updating Multivariate Oyster Population Model.

FIG. 4 illustrates an exemplary method to build a system for creating Multivariate Oyster Population Models.

FIG. 5 is a table containing exemplary function parameters which may be stored and updated to create a Multivariate Predictive Model.

FIG. 6 is an exemplary data structure which contains sample attribute values for an instance of Reef Object.

FIG. 7 is an exemplary data structure which contains sample attribute values for an instance of an Oyster Group Demographic Object.

FIGS. 8a through 8k illustrate exemplary data structures for Multivariate Oyster Population Models.

FIG. 9 illustrates in which field data is processed and stored for retrieval during the creation of a multivariate Oyster Population Model.

FIGS. 10a through 10g illustrate exemplary Multivariate Oyster Population Models.

### TERMS OF ART

As used herein, the term “associate,” “associated” and “association” means a relationship which may be expressed as a value or parameter, and used for navigation, search, retrieval, updating, instantiation, operations or for invoking functions.

As used herein, the term “attribute” means a characteristic, feature, or state represented by a numeric value; an attribute value may be updated by functions, and changes in attribute values may, in turn, invoke functions.

As used herein, the term “attribute category” means one or multiple attributes which may be functionally, categorically or conceptually related. An attribute category may refer to one or multiple attributes.

As in used herein, the term “class” means a processing component having functional processing capability for creating instances objects (which are also processing components) having common attributes and or functions. Classes and objects may operate as virtual machines.

As used herein the “computer architecture” or “server” means an integrated set of processing components which define the specialized functionality of a computer apparatus or network; computer architecture or server components include, but are not limited to hardware components, data structures, class and object definitions, virtualized components and/or components stored in memory which are non-modifiable at run time to emulate physical hardware components.

As used herein, the term “data” or “data structure” is any data in any format which can be stored in a computer and which may include non-modifiable attributes and values once created.

As used herein, the term “field-data values” means values obtained or derived from experimentation or observation.

As used herein, the term “growth value” means any attribute, value, or mathematical expression of growth.

As used herein, the term harvest size means any attribute, value, or mathematical expression of quantity harvested.

As used herein the term “harvest size” means any attribute, value, or mathematical expression expressing a metric related to a harvest.

As used herein, the term “instantiating” or “instantiation” means the creation of an instance of a processing component, class, object or other data structure.

As used herein the term “invoke” means to initiate or call a function or an operation which causes a physical change or transformation.

As used herein, a “look-up table” or “table” refers to a data structure which stores data in an associative manner including an indexed table, hash table, multi-level array, or grid; in various embodiments. A table may be an indexed structure that replaces computations with an indexed value that is retrieved using a “look up” function.

As used herein, a “meta-analysis” or “meta-analysis function” means method which may be expressed as a function which combines statistical functions or processes, and which, in various embodiments, sequences, variables and/or weighting to provide a Multivariate Model with the least amount of error.

As used herein, the term “model” means a digital representation of an entity for phenomena which includes that which may be updated continuously, sporadically, or in real time.

As used herein, the term “multivariate” means reflecting observation, calculation, revision, storage, retrieval, or analysis of more than one attribute, parameter, variable, or combination thereof relevant to a representation or outcome.

As used herein, the term “Multivariate Predictive Model” means one or more files, data structures, or objects reflecting the multivariate analysis for any State Model or for purposes of predicting an outcome.

As used herein, the term “object” means an instance of a class which represents an entity for tracking; objects have attributes and functions and may operate as separate processing components or virtual machines.

As used herein, the term “oyster group demographic attribute” means any attribute of an Oyster Group Demographic Object that can be mathematically expressed, including but not limited to larval dispersal, age at first reproduction, stage specific mortality, fecundity, identify, age, life state, sex, size, shell gain, energy reserves, biomass, reef association, natal reef number, reproductive status, and patch state variables.

As used herein, the term “Oyster Group Demographic Object” means a processing component with attributes and processing functions to represent or model an oyster demographic group population state under a particular set of parameters or scenario; an object independent processing capability.

As used herein, the term “parameter” means an attribute and the associated attribute value.

As used herein, the term “parameterization function” means a function to calculate or update a parameter.

As used herein, the term “processor” “processing component” means a microprocessor or other hardware component having processing capability which may be bound to non-modifiable values an and functions.

As used herein, the term “Project” means a file, object, memory storage location, or other data structure known in the art which contains data relevant to specific study parameters. In various embodiments, a Project may be an object.

As used herein, the term “real time” means during a user session, or any time period allocated for study and analysis.

As used herein, the term “Reef Object” means an object which represents a population of one or more oysters having at least one common attribute, and which may or may not include functions and processes which may be invoked when attributes are populated or updated, causing the Oyster Group Demographic Object to function as a separately identifiable processing component.

As used herein the “server” or “computer architecture” means an integrated set of processing components which define the specialized functionality of a computer apparatus or network; computer architecture or server components include, but are not limited to hardware components, data structures, class and object definitions, virtualized components and/or components stored in memory which are non-modifiable at run time to emulate physical hardware components.

As used herein, the term “spawn values” means a number or value related to the number or rate of spawn produced.

As used herein, the term “State Model” or “State Model Function,” means reflecting a state under a scenario or point in time.

As used herein, the term “survivor values” or “survival rate” means a number or value related to the rate of survival.

As used herein, the term “user input” or “input” means data, variables, and parameters entered, input, or retrieved by a user and imported from an external source, or retrieved from an existing model, object, or storage location in the computer. Examples of user-defined parameters include but are not limited to spatial scale, time step, length of simulation, depth, temperature duration, salinity, salinity duration, TSS, TSS duration, dissolve oxygen, initial oyster biomass, and initial oyster density.

As used herein, the term “virtualized” or “virtualized components” means software which simulates or assumes the functionality of hardware.

### DETAILED DESCRIPTION OF THE INVENTION

The following description of exemplary embodiments of a Multivariate Predictive Modeling System shall be interpreted with reference to U.S. Supreme Court standards pertaining to computer implemented inventions. Functional processing components may be described in terms of hardware or software processing (“virtual”) components. The term “apparatus” may refer to one or multiple devices and may contain virtual components functionally integrated with hardware to perform novel or specialized processing functions. Furthermore, various types of virtual components may be referred to as “classes” or “objects,” however this designation shall not be construed as language or platform specific. A class, object or virtual component may refer to any aggregation of functions and data types which may be functionally bound to a microprocessor to form a specific purpose computer with novel and identifiable capabilities.

The terms “a” and “an” may refer to a single or multiple elements of the same type and shall be interpreted as “at least one.” The term “plurality” shall mean two or more. Steps may be performed in any order and shall be construed to encompass any function, formula, process or transformative action.

References to data types and data sets (e.g. attributes, parameters and variables) shall be interpreted as data sets derived through experimentation to yield specific or unexpected results. Tables may be identified as representing data structures, arrays.

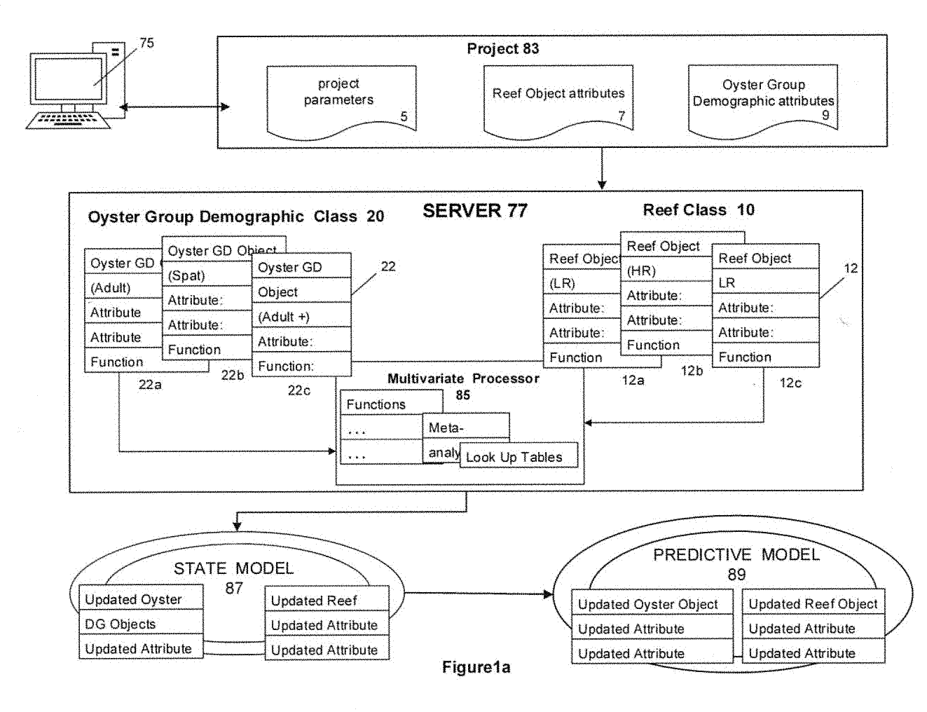

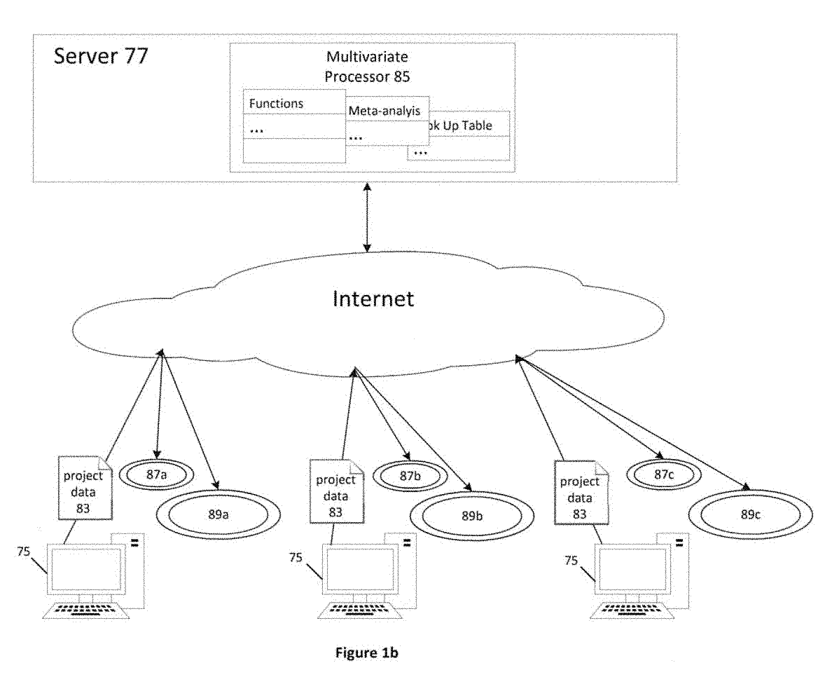

FIG. 1a illustrates an exemplary Multivariate Predictive Modeling System 100 which is implemented on a computer architecture that includes classes and objects that operate as virtual machines.

In the exemplary embodiment shown, Multivariate Predictive Modeling System 100 includes user interface 75 configured to receive various types of project data from a user interface 75 or other external source to instantiate Project 83.

In various embodiments, Project 83 may be implemented an object, file, data structure, or internal or external memory storage area within Multivariate Predictive Modeling System 100. Project 83, if implemented as a class or object, may perform or invoke other functions, such as instantiating classes and objects.

Project data may be any parameter, argument, value, data or code sequence known in the art, and is not limited to data within the depicted categories.

In the exemplary embodiment shown, project data includes, project parameters 5, reef attributes 7 and oyster group demographic attributes 9, which are used to instantiate Project 83,

In other embodiments, project parameters 5 include geographical parameters to define a geographical area containing one or more reefs and a time period to define the duration for study, analysis, modeling and observation. Project 83 may include multiple time parameters identifying multiple time periods over which outcomes are to be predicted, and during which project or system functions may be invoked and/or sequentially, repetitively, iteratively or recursively run during the time intervals and parameters.

In various embodiments, user-defined or predetermined project parameters 5 may include, but are not limited to, number of reefs time step, length of simulation. H2O temperature, temperature duration, salinity duration, TSS duration, and dissolved oxygen (DO) duration.

Project parameters 5 may identify any metric or characteristic which may be expressed and/or tracked using a mathematical representation or numeric value.

Project data further includes reef attributes 7 to instantiate one or more Reef Objects 12a, 12b and 12c. Reef Objects 12a, 12b and 12c represent the identity and characteristics of one or more reefs located within the geographical parameters defined by Project 83, and are configured with reef object functions enabling each Reef Object 12a, 12b and 12c to independently perform calculations and processes to update reef object attributes.

Reef object attributes identify and reflect properties of a particular reef within a project location (e.g. for tracking and/or study). Reef object attributes may identify any reef metric or characteristic which may be expressed and/or tracked using a mathematical representation or numeric value.

Project data further includes oyster group demographic attributes 9, which are statistically derived values representing observed, estimated or statistically calculated characteristics of a defined oyster demographic group under study.

Exemplary oyster group demographic attributes 9 include but are not limited to: initial oyster biomass, age at first reproduction, stage specific mortality, fecundity, reproduction, status, identity (what reef), life stage, sex, size (total shell growth), shell gain—rate of growth (daily shell growth), energy reserves (expenditure of 1.2 unit/day). energy reserves (influenced by environment) biomass, location of natal reef, reproductive status, current reef location, and patch state variables.

In various embodiments Project Server 77 includes Reef Class 10 and Oyster Group Demographic Class 20 which are processing components configured with class functions to instantiate Reef Objects 12a, 12b, 12c, and Oyster Group Demographic Objects 22a, 22b and 22c.

Reef Objects 12a, 12b, and 12c include attributes to identify and reflect properties of a particular reef within a project location (e.g. for tracking or study). Reef object attributes may identify any reef metric or characteristic which may be expressed and/or tracked using a mathematical representation or numeric value.

Oyster Group Demographic Objects 22a, 22b and 22c represent oyster demographic groups with specially selected attributes and functions received as inputs or calculated by invoking Oyster Group Demographic Object functions which allow Oyster Group Demographic Objects 22a, 22b and 22c to function as independent processing components.

In the exemplary embodiment shown, Server 77 includes or is operatively coupled with Multivariate Processor 85. Multivariate Processor 85 invokes system functions, project functions and object functions to create at least one State Model 87 to reflect a baseline state (or other desired state) having attribute values reflective of oyster populations demographics within reefs and/or geographical locations under study.

In various embodiments, system functions, project functions and object functions may be called or selected by a user, or invoked when objects or parameters are initialized or changed. In various embodiments, system functions, project functions and object functions may be combined and modified to create meta-analysis tools and to perform multivariate calculations. System functions, project functions and object functions may include retrieval of data values from internal look up tables, hash tables or other data structures, or from external data bases.

In various embodiments, Multivariate Processor 85 may update or modify attributes of Reef Objects 12a, 12b, 12c and Oyster Group Demographic Objects 22a, 22b and 22c to create one or more State Models 87 or Multivariate Predictive Models 89 which reflect attributes of multivariate factors on oyster populations under different scenarios.

In various embodiments, functions performed by Multivariate Processor 85 may compare attributes of multiple State Models 87 and/or Multivariate Predictive Models 89 to identify relevant correlations and patterns.

Multivariate Processor 85 may utilize functions to alter functions, parameters, and arguments to combine conceptually similar scientific studies to standardize or normalize and standardize study parameters, calculations and methodologies. In various embodiments, a Multivariate Processor 85 may calculate values reflected in a Multivariate Predictive Model 89 by utilizing functions for weighting calculations or generating approximations. Functions for weighting and approximation may be standardized for various embodiments of a Multivariate Predictive Model 89.

In various embodiments, Multivariate Processor 85 may be configured to identify inconsistencies and errors in the context of multiple studies or field data sets.

In various embodiments, Multivariate Processor 85 may be configured to receive user-selected or user defined functions or data sets, or may allow a user to exclude data or functions derived from specific studies.

FIG. 1b illustrates geographically distributed embodiment of Multivariate Predictive Modeling System 100 which is remotely accessed multiple users to create Predictive Models. As illustrated in FIG. 1b, one or more Servers 77, which include one or more Multivariate Processors 85, are accessed by one or more User Interfaces 75 to create State Models 87a, 87b and 87c, and Multivariate Predictive Models 89a, 89b and 89c. In various embodiments, users may enter field data or hypothetical values, and select customized combinations and sequences of modeling functions, multivariate functions and meta-analysis functions. Various embodiments may allow users to select pre-programmed sequences of functions (templates) to represent particular outcomes as State Models 87a, 87b and 87, and Multivariate Predictive Models 89a 89b and 89c.

In various embodiments functions and standardized parameter sets may be stored in look-up tables, which may be linked or associated with particular studies, or indexed as stored values to import. In various embodiments users may access data or data sets from particular studies and elect to exclude data or functions from particular studies, and/or select among alternative methodologies. In other embodiments, functions may be selected which combine and/or weight the results of multiple functions. Various embodiments may allow users to access features which alter the parameters or sequence of functions, weight the parameters or normalize them to allow various functions to be combined to produce State Models 87a 87b and 87c and Multivariate Predictive Models 89a, 89b and 89c multivariate and meta-analysis relative to oyster population impacts, metrics and outcomes.

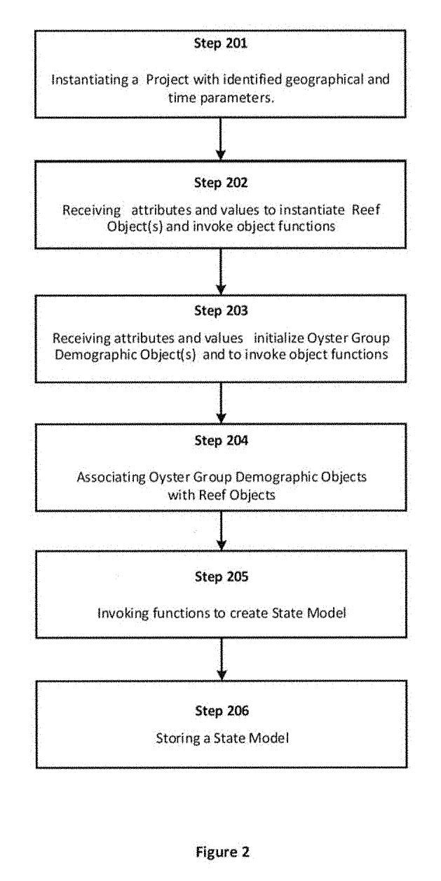

FIG. 2 illustrates an exemplary method for creating a Multivariate Oyster Population Model reflecting a baseline state.

Step 201 is the step of receiving input to instantiate a Project with geographical, time, and other parameters.

Step 202 is the step of receiving defined attributes and values to instantiate and initialize Reef Object(s) associated with a project and to invoke Reef Object Functions

Step 203 is the step of receiving defined attributes and values to instantiate and initialize Oyster Group Demographic Object(s) associated with a project and to invoke Oyster Group Demographic Object Functions.

Step 204 is the step of instantiating Oyster Group Demographic Objects and associating with a Reef Object.

Step 206 is the step of selecting and invoking functions to create a State Model.

Step 207 is the step of storing a State Model which may be used as baseline.

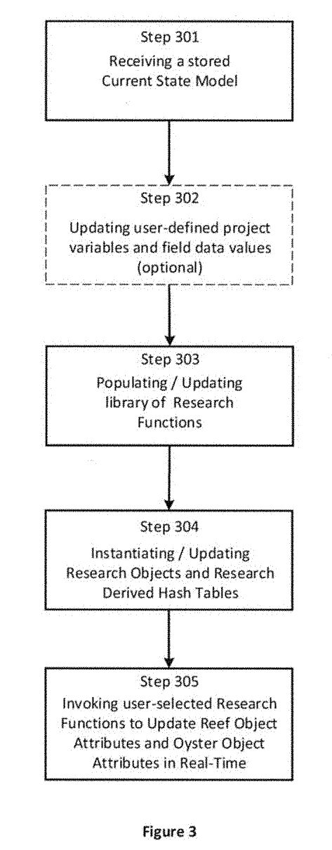

FIG. 3 illustrates an exemplary method for updating a Multivariate Oyster Population Model.

Step 301 is the step of receiving a stored State Model.

Step 302 is the step of updating user-defined project variables and field data values (optional) to reflect alternate scenarios.

Step 303 is the step of populating function parameters.

Step 304 is the step of instantiating and/or updating research objects and hash tables which may be used for accessing and storing function values.

Step 305 is the step of invoking user-selected research functions to update reef object Attributes and Oyster Object Attributes in real time.

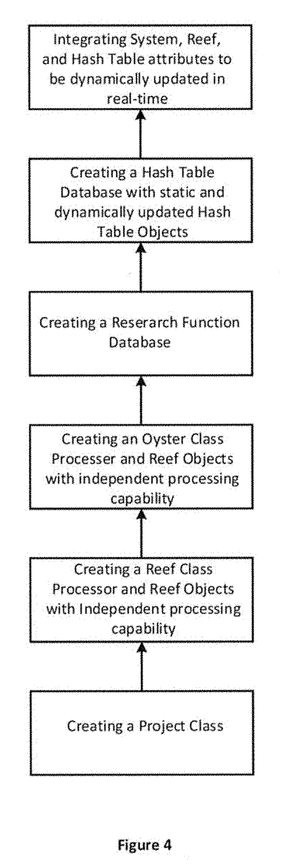

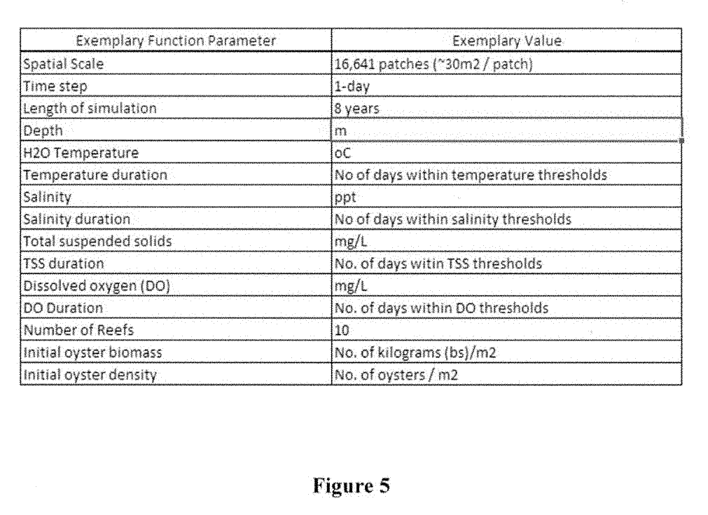

FIG. 4 illustrates an exemplary method for building a computer system for creating Multivariate Oyster Population Models.

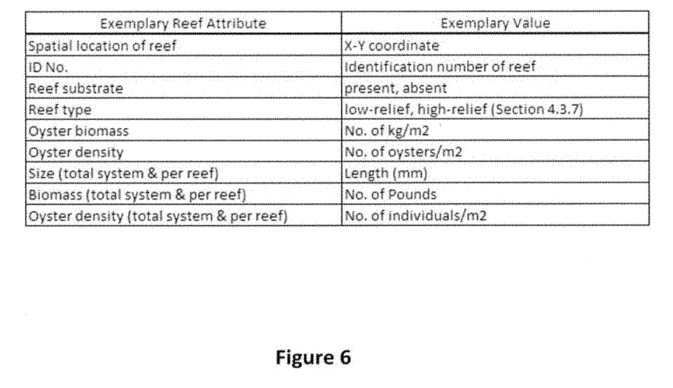

FIG. 6 is a table containing exemplary function parameters which may be stored and update create a Multivariate Predictive Model. In the exemplary embodiment shown, the function parameters include a time step, length of simulation, H2O temperature, temperature duration, salinity duration, TSS duration, and DO duration.

FIG. 6 is an exemplary data structure which contains sample attribute values for an instance of Reef Object. These exemplary attributes shown in FIG. 6 includes: spatial scale, depth, H2O temperature, salinity, TSS, dissolved oxygen (DO), initial oyster density, larval dispersal, spatial location, ID number, reef substrate, reef type, oyster biomass, oyster density, age distribution of oysters, ‘adult+’ oysters, adult oysters, sub-adult oysters, spat/juvenile oysters, total population size, proportion of ‘adult+’s, proportion of adults, proportion of sub-adults, proportion of spat/juveniles, size (total system and per reef), biomass (total system and per reef), and oyster density (total system and per reef).

Reef object attributes identified in FIG. 6 are exemplary, may identify any reef metric or characteristic which may be expressed and/or tracked using a mathematical representation or numeric value.

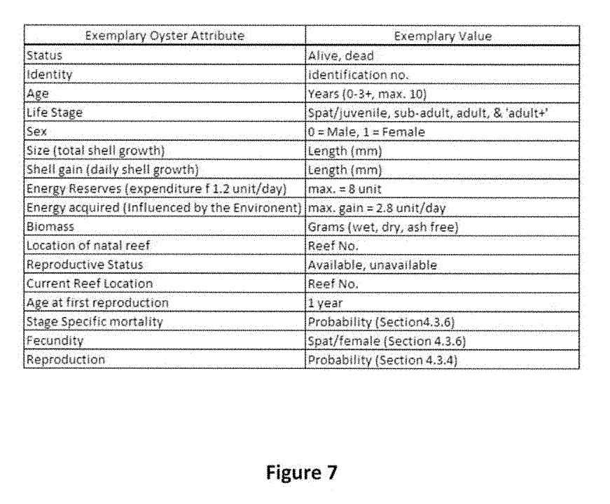

FIG. 7 is an exemplary data structure which contains sample attribute values for an instance of an Oyster Group Demographic Object. Oyster attributes include but are not limited to: initial oyster biomass, age at first reproduction, stage specific mortality, fecundity, reproduction, status, identity (what reef), life stage, sex, size (total shell growth), shell gain—rate of growth (daily shell growth), energy reserves (expenditure of 1.2 unit/day), energy reserves (influenced by environment) biomass, location of natal reef, reproductive status, current reef location, and patch state variables. Oyster group demographic attributes identified in FIG. 6 are exemplary, may identify any oyster or oyster population related metric or characteristic which may be expressed and/or tracked using a mathematical representation or numeric value.

In various embodiments, oyster group demographic attributes reflect phases of a biphasic life cycle (i.e., sessile adult and motile larval stages), and changes in this attributes may invoke functions to perform statistical calculations of viability based environmental factors including, but not limited to, flow regime, TSS, temperature, salinity, and dissolved oxygen.

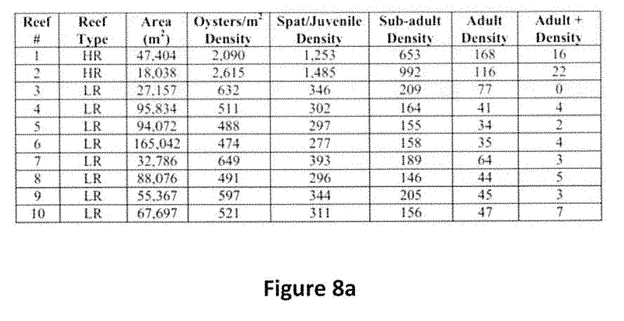

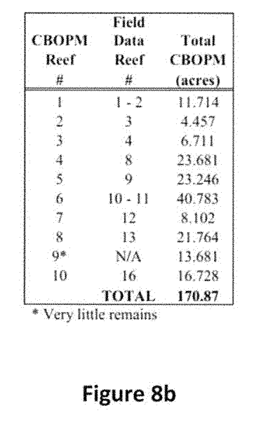

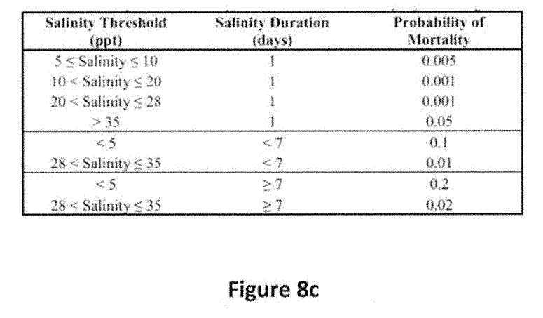

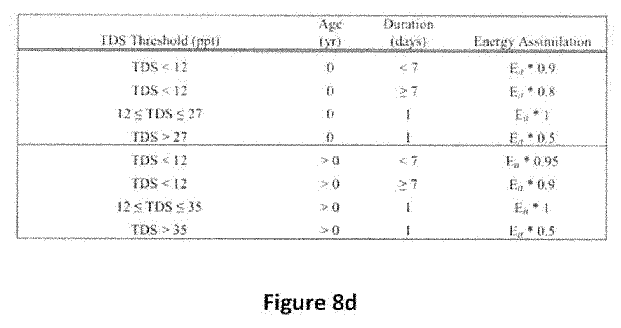

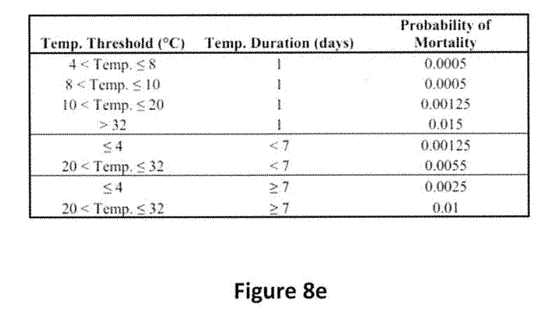

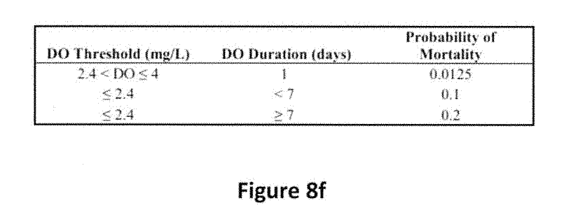

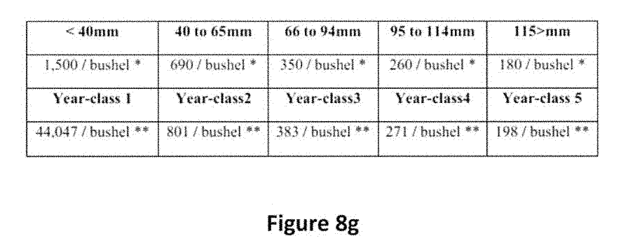

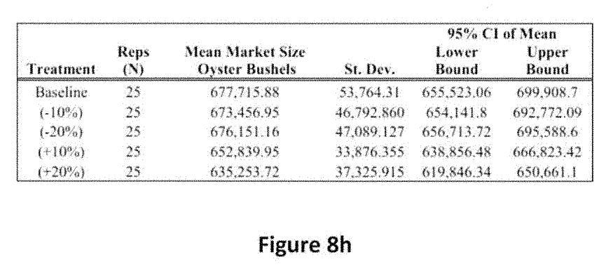

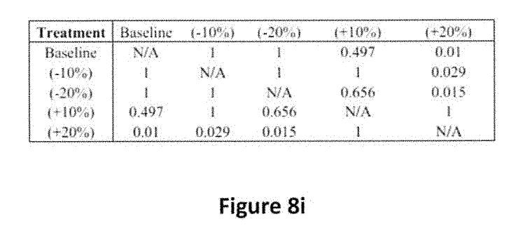

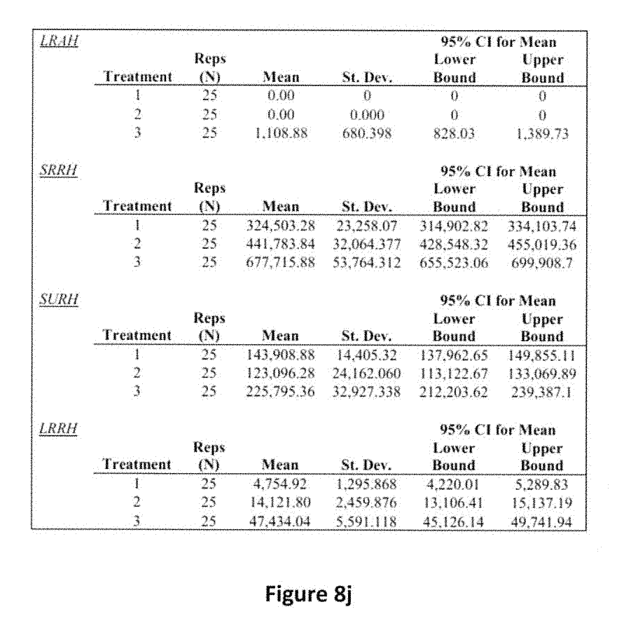

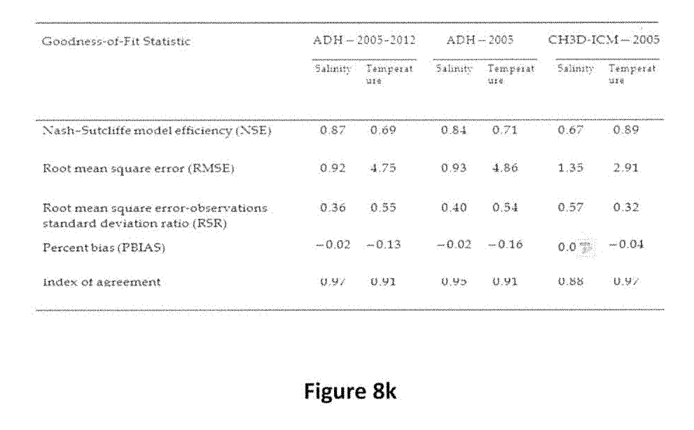

FIGS. 8a through 8k illustrate exemplary data structures, included look-up tables, which store values and parameters used by functions invoked Multivariate Processor functions called by classes and objects to produce Multivariate Predictive Models. FIG. 8a is an exemplary data structure which stores attributes values for reefs, including reef type, area, oyster density, spat/juvenile density, sub-adult density and adult+ density. FIG. 8b is a look-up table storing values and identifiers for independent reefs and their acreage. FIG. 8c is an exemplary look-up table which stores values or probability of mortality based on salinity threshold and duration of salinity. FIG. 8d is a look-up table from which correlate values may be accessed by total duration salinity, age, duration and energy assimilation. FIG. 8e illustrates the probability of mortality based on temperature threshold and temperature duration. FIG. 8f is an exemplary data structure which indexes probability of mortality based on dissolved oxygen threshold and dissolved oxygen duration. FIG. 8g is an exemplary data structure which correlates or indexes bushel harvest values based on shell length and shell class. FIG. 8h indexes values mean market size of oyster bushels based on treatment, with standard deviation and upper/lower bounds. FIG. 8i a look-up table reflecting a correlation between a matrix of treatments. FIG. 8j is a data structure which stores correlated values for a harvest based on treatment and harvest type. FIG. 8k illustrates a data structure from goodness of fit statistics may be accessed.

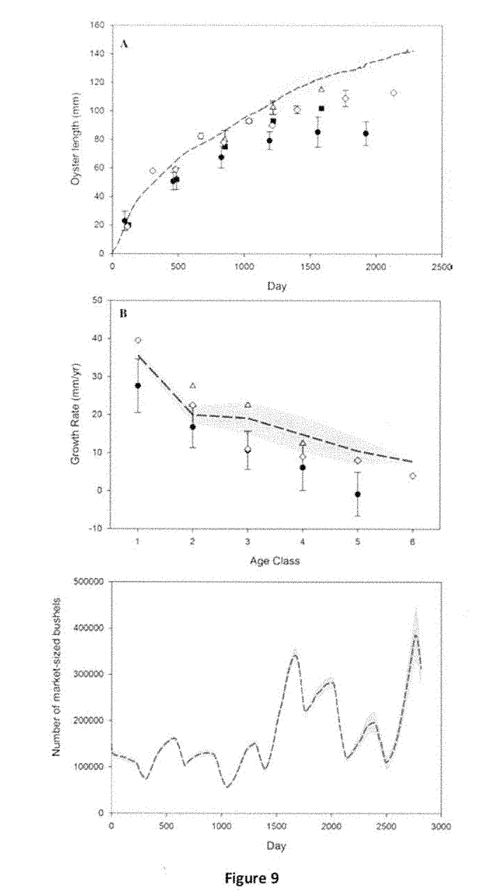

FIG. 9 is an exemplary Multivariate Oyster Population Model which combines attributes from multiple State Models and/or Multivariate Predictive Models and represents them graphically over time. In the exemplary embodiment shown, graph A shows oyster length by treatment group over time. Graph B shows growth rate by age class. Graph C shows number of bushels over time.

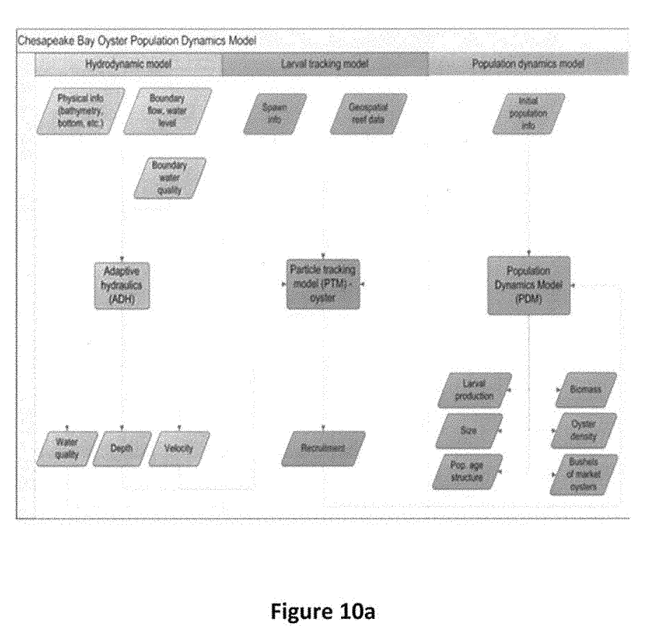

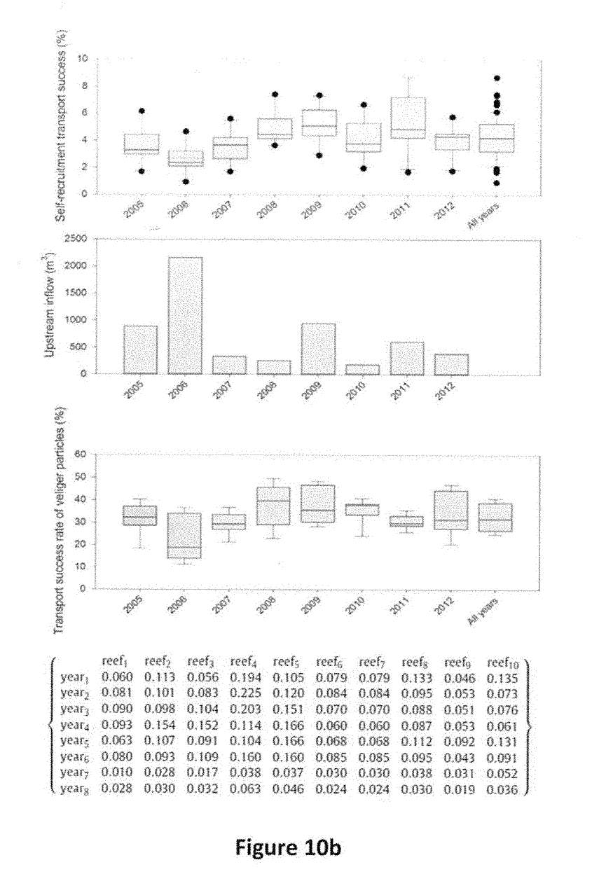

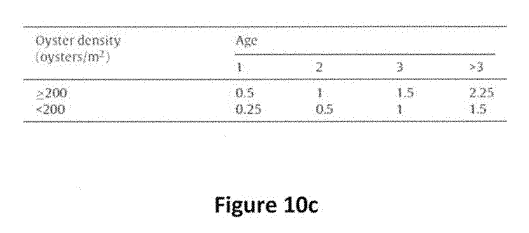

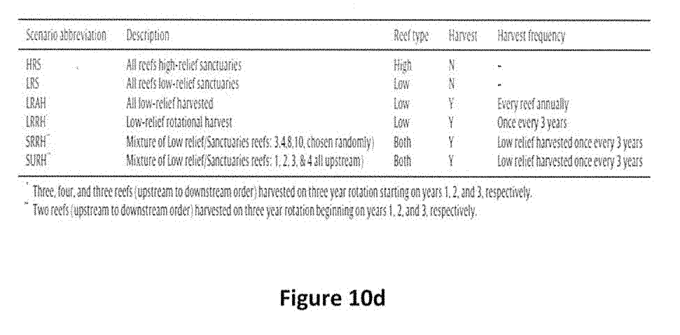

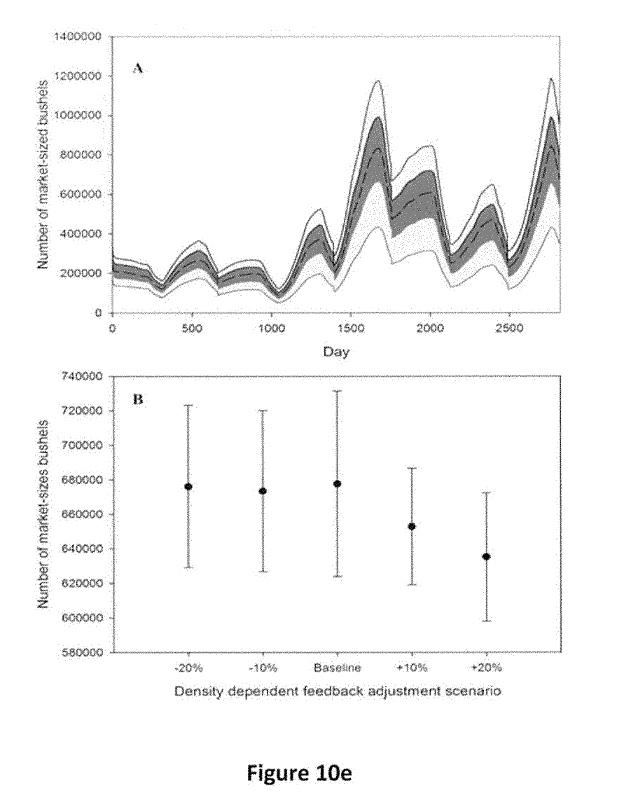

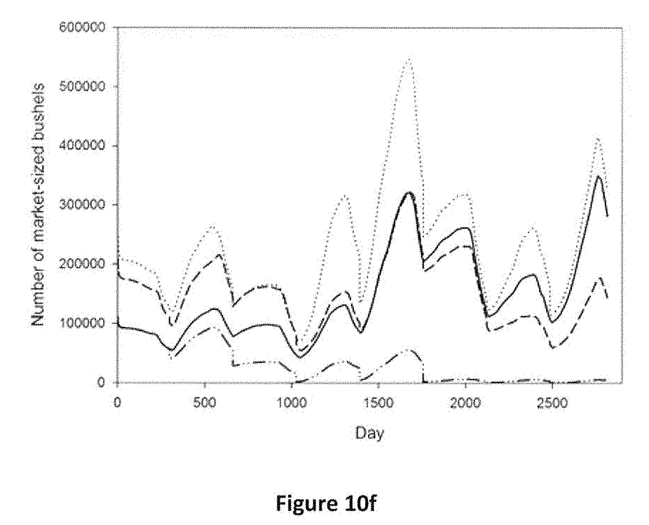

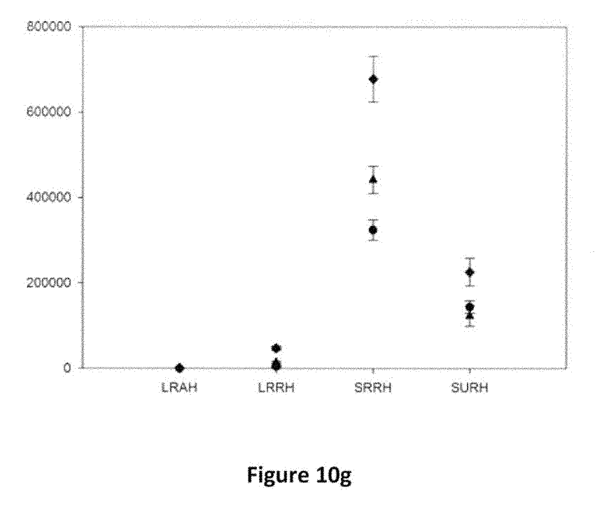

FIGS. 10a through 10g illustrate several exemplary Multivariate Oyster Population Models.

FIG. 10a illustrates a graphical representation of a Multivariate Oyster Population Models which incorporates three models: a hydrodynamic model (left panel), Larval tracking model (middle panel) and a spatially-explicit agent based population dynamics model (right panel). Initialization requirements are displayed in the top row, the specific models used are located in the middle, and the bottom row represents the outputs of each model. Arrows indicate the directions of input/output linkages among the models.

FIG. 10b illustrates a Multivariate Oyster Population Models (A) PTM self-reflecting recruitment across 8 years. Dotted lines indicate min and max rates and dots represent statistical outliers; (B) summer freshwater inflow volume; and (C) transport success rate of veliger particles across 8 years.

FIG. 10c illustrates the effect of reef density (RDE) on age-specific fecundity values.

FIG. 10d illustrates a description of the scenarios tested using the Chesapeake Bay Oyster Population Model (CBPOM).

FIG. 10e is an exemplary Multivariate Predictive Model that is based on 25 stochastic replicates. (A) Comparison of changes in the number of market-sized bushels of the baseline scenario (dotted line) to scenarios when initial oyster density was increased or decreased by 50% (light gray) or 25% (Dark gray). (B) Comparison of the final number of market-sized bushels, after an eight-year simulation, of the baseline scenario (dotted line) to scenarios when the density dependent feedback factor was altered by ±10% or 20%.

FIG. 10f illustrates an exemplary Multivariate Predictive Model reflecting market-sized bushels under management strategies under different harvest regimes. The exemplary embodiment shown illustrates four randomly placed sanctuary reefs and six rotationally harvested reefs under high reef and low reef scenarios. The exemplary Multivariate Predictive Model shown reflects scenarios of ten rotationally harvested reefs, with varied parameters, and varying low and high reef parameters.

FIG. 10g is an exemplary Multivariate Predictive Model of harvest outcomes under alternative scenarios in which harvest limit parameters have been adjusted.

1. An apparatus comprised of:
a server configured to receive project parameter values, reef parameter values and oyster group demographic parameter values;
wherein said server includes virtual machine components including;
a Project Instantiation Processor which is a Project Class;
a Reef Instantiation Processor which is a Reef Class; and
an Oyster Group Demographic Instantiation Processer which is an Oyster Group Demographic Class.

2. The apparatus of claim 1 wherein said Project Instantiation Processor is configured to receive geographical parameters, time parameters and reef association parameters.

3. The apparatus of claim 1 wherein said Project Instantiation Processor is configured to instantiate at least one Project Object.

4. The apparatus of claim 3 wherein said at least one Project Object includes project attributes, functions, function parameters and project parameters to pass values to said Reef Instantiation Processor.

5. The apparatus of claim 1 wherein s Reef Instantiation Processor is configured to instantiate at least one Reef Object.

6. The apparatus of claim 5 wherein each of said at least one Reef Object is associated with a Project Object.

7. The apparatus of claim 5 wherein each said at least one Reef Object is comprised of reef attributes and reef attribute processing functions.

8. The apparatus of claim 6 wherein said at least one Reef Object is further configured with functions to send a signal to said Oyster Group Demographic Instantiation Processor to instantiate at least one Oyster Group Demographic Object.

9. The apparatus of claim 8 wherein each of said at least one Oyster Group Demographic Object is associated with said at least one Reef Object.

10. The apparatus of claim 8 wherein each said at least one Oyster Group Demographic Object has oyster group demographic attributes and oyster group demographic attribute processing functions.

11. The apparatus of claim 3 wherein each of said at least one Project Objects is further configured to invoke functions to produce and store at least one Multivariate Predictive Model.

12. The apparatus of claim 1, which further includes a Function Library which is configured with functions having parameters which may be initialized and updated.

13. The apparatus of claim 12 wherein said Function Library includes at least one function set data structure which identifies functions which are invoked to create and update reef attributes and oyster group demographic attributes to create a Multivariate Predictive Model.

14. The apparatus of claim 13 wherein said Multivariate Predictive Model is a State Model which receives updated parameters for the functions invoked by said a least one function set data structure.

15. The apparatus of claim 3 wherein said at least one Project Object further includes at least one State Models.

16. The apparatus of claim 1 which further includes at least one Multivariate Predictive Model.

17. The apparatus of claim 8, wherein changes in values associated with attributes of said at least one Oyster Group Demographic Object invokes functions to update associated Reef values.

18. The apparatus of claim 8 wherein said server includes a processor to extract and compare attributes from a plurality of Oyster Group Demographic Objects.

19. The apparatus of claim 5 wherein said server includes a processor to extract and compare attributes from a plurality of Reef Objects,

20. The apparatus of claim 1 which further includes Project Objects that are Multivariate Oyster Population Models.

21. The apparatus of claim 20 wherein attributes of said Multivariate Oyster Population Modes are updated in real time.
